(19) United States
(12) Patent Application Publication
(10) Pub. No.: US 2025/0250547 A1
(43) Pub. Date: Aug. 7, 2025

(54) OPTIMIZATION OF HEK293 SUSPENSION PLATFORM FOR IMPROVED rAAV TITERS

(71) Applicant: Adverum Biotechnologies, Inc., Redwood City, CA (US)

(72) Inventor: Ali Aghajanirefah, Redwood City, CA (US)

(73) Assignee: Adverum Biotechnologies, Inc., Redwood City, CA (US)

(21) Appl. No.: 18/855,209

(22) PCT Filed: Apr. 7, 2023
(86) PCT No.: PCT/US2023/017838

§ 371 Date: Oct. 8, 2024

Related U.S. Application Data
(60) Provisional application No. 63/331,438, filed on Apr. 15, 2022.
(60) Provisional application No. 63/329,856, filed on Apr. 11, 2022.

(51) Int. Cl. C12N 7/00

(52) U.S. Cl. CPC C12N 7/00; C12N 2750/14122; C12N 2750/14141; C12N 2750/14152

(57) ABSTRACT

The invention provides methods and systems for production of high-titer rAAV. The high-titer rAAV may be used in pharmaceutical compositions, gene therapy and research.

### CROSS-REFERENCE TO RELATED APPLICATIONS

This application is a 371 National Stage filing and claims the benefit under 35 U.S.C. § 120 of International Application No. PCT/2023/017838, filed Apr. 7, 2023, which claims priority under 35 U.S.C. § 119 (e) to U.S. Provisional Application No. 63/329,856 filed Apr. 11, 2022, and U.S. Provisional Application No. 63/331,438 filed Apr. 15, 2022; each of which is incorporated by reference in its entirety.

### STATEMENT REGARDING THE SEQUENCE LISTING

The official copy of the Sequence Listing is submitted concurrently with the specification as an WIPO Standard ST.26 formatted XML file with file name “17234-037WO1_SequenceListing.xml”, a creation date of Apr. 6, 2023, and a size of 4,096 bytes. This Sequence Listing filed via USPTO Patent Center is part of the specification and is incorporated in its entirety by reference herein.

### FIELD OF INVENTION

The present invention relates to the field of production of recombinant AAV.

### BACKGROUND

AAV is a 4.7 kb, single-stranded DNA virus. rAAV virions are associated with excellent clinical safety, since wild-type AAV is non-pathogenic and has no etiological association with any known disease. Recent clinical and non-clinical success with recombinant adeno-associated virus (rAAV) vector as an in vivo gene delivery vehicle has increased interest in producing high purity and efficacious viral vectors for various natural and engineered serotypes. AAV requires expression of helper genes for efficient replication The current rAAV production in human embryonic kidney (HEK) 293 cells system includes helper plasmid providing the helper genes instead of helper virus and is thus referred as a helper free system.

AAV vectors used for administration of therapeutic nucleic acids typically have approximately 96% of the parental genome deleted, such that only the terminal repeats (ITRs) which contain recognition signals for DNA replication and packaging remain. This reduces immunologic or toxic side effects due to expression of viral genes. In addition, delivering specific AAV proteins to producing cells enables integration of the AAV vector comprising AAV ITRs into a specific region of the cellular genome if desired. See, for example, U.S. Pat. Nos. 6,342,390 and 6,821,511. Host cells comprising an integrated AAV genome show little or no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex.

Several AAV serotypes and over 100 AAV variants have been isolated from adenovirus stocks or from human or non-human primate tissues. Generally, the AAV serotypes have genomic sequences of significant homology at the nucleic acid sequence and amino acid sequence levels, such that different serotypes have an identical set of genetic functions, produce virions which are physically and functionally equivalent, and replicate and assemble by nearly identical mechanisms. AAV serotype 2 (AAV2) has been used extensively for gene therapy applications.

AAV Rep and ITR sequences are particularly conserved across many AAV serotype. Moreover, the Rep sequences and ITRs of many AAV serotypes are known to efficiently cross-complement (i.e., functionally substitute) corresponding sequences from other serotypes during production of AAV particles in mammalian cells. Generally, the Cap proteins, which determine the cellular tropicity of the AAV particle and related Cap protein-encoding sequences, are significantly less conserved than Rep genes across different AAV serotypes. In view of the ability of Rep and ITR sequences to cross-complement corresponding sequences of other serotypes, the AAV vector can comprise a mixture of serotypes and thereby a “chimeric” or “pseudotyped” AAV vector. A chimeric AAV vector comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotypes are further described in for example U.S. Pat. No. 6,723,551; Flotte (2006) Mol Ther 13 (1): 1-2; Gao et al (2004) J. Virol 6381-6388; Gao et al (2002) Proc Natl Acad Sci USA 99:11854-11859; De et al (2006) Mol Ther 13:67-76, and Gao et al (2006) Mol Ther 13:77-87.

The most commonly used method for production of recombinant adeno-associated virus (rAAVs) in research laboratories is by transient triple transfection of 293 cells with AAV cis and trans plasmids and an adenovirus helper plasmid. The triple transfection HEK293 platform is routinely used for rAAV production and utilizes an AAV cis plasmid encoding the gene of interest, an AAV trans plasmid comprising capsid and replicase genes, and a helper plasmid. However, most of the currently used HEK293 manufacturing platforms yield low titers and are not scalable.

### SUMMARY

Methods and systems of producing high-titer rAAV are provided herein. The rAAV produced by these methods and systems may be used in pharmaceutical compositions, gene therapy compositions, for research purposes and for other uses.

In an embodiment, a method producing high-titer rAAV comprising the steps of providing HEK293-derived cells, co-transfecting the HEK293-derived cells with plasmids comprising an ITR expression vector, an AAV helper construct, Rep and Cap, incubating the transfected cells and harvesting the rAAV is provided. In an aspect of the method, Rep and Cap are on separate plasmids. In an aspect, Rep and Cap are on the same plasmid. In aspects of the method, the transfection reagent used in co-transfecting the cells is selected from the group comprising AAV-Max and PEI-Pro. In various aspects, the HEK239 derived cells are selected from the group comprising VPC2.0 and Oxford cells. In an aspect, the HEK293-derived cells are selected from the group comprising VPC2.0 cells and Oxford cells and wherein the transfection reagent used in co-transfecting the cells is selected from the group comprising AAV-Max and PEI-Pro. In aspects of the method, the HEK293-derived cells are VPC2.0 cells and the transfection reagent is AAV-Max. In an aspect, the titer of the harvested rAAV is at least 5×10¹⁰ vg/ml. In various aspects, the transfection reagent used in co-transfecting the cells is selected from the group comprising AAV-Max and PEI-Pro, and Rep and Cap are on separate plasmids. In various aspects, the transfection reagent used in co-transfecting the cells is selected from the group comprising AAV-Max and PEI-Pro, and Rep and Cap are on the same plasmid. In an aspect the transfection reagent used in co-transfecting the cells is AAV-Max, and Rep and Cap are on separate plasmids. In an aspect the transfection reagent used in co-transfecting the cells is TRX, Rep and Cap are on separate plasmids, and the HEK293-derived cells are VPC2.0 cells. In various aspects, the HEK293-derived cells are selected from the group comprising VPC2.0 cells and Oxford cells, the transfection reagent used in co-transfecting the cells is selected from the group comprising AAV-Max and PEI-Pro, and Rep and Cap are on separate plasmids. In some aspects, the HEK293-derived cells are VPC2.0 cells, the transfection reagent is AAV-Max and Rep and Cap are on separate plasmids. In various aspects the titer of the harvested rAAV is at least 1×10¹¹ vg/ml.

A four plasmid system for producing high titer rAAV is provided. The four plasmid system for producing high titer rAAV comprises the steps of providing a set of plasmids comprising a first plasmid comprising a nucleotide sequence encoding an AAV Rep polypeptide operably linked to a first promoter, a second plasmid comprising a nucleotide sequence encoding an AAV Cap polypeptide operably linked to a second promoter, a third plasmid comprising an ITR expression vector comprising a gene of interest, and a fourth plasmid comprising an AAV helper construct, transfecting HEK293-derived cells with the set of plasmids, incubating the transfected cells, lysing the transfected cells and harvesting the rAAV. In aspects of the system, the second promoter is a constitutively active promoter. In certain aspects, the constitutively active promoter is a CMV promoter. In aspects of the system, the second promoter is a p40 promoter. In aspects of the system, the first promoter is an AAV promoter. In certain aspects, the AAV promoter is selected from the group comprising a p5 promoter and a p19 promoter. In aspects of the system, the HEK293-derived cells are selected from the group comprising HEK293 cells, VPC2.0 cells and Oxford cells. In aspects of the system, the molar ratio of the first plasmid to the third plasmid and the molar ratio of the second plasmid to the third plasmid are comparable when normalized to plasmid size. In various aspects, the four plasmids of the set of plasmids are co-transfected in molar ratios of approximately 1:1:1:1 when normalized to plasmid size. In various aspects, the molar ratio of the first plasmid to either the third plasmid or the fourth plasmid is greater than 1:1, when normalized to plasmid size. In various aspects, the molar ratio of the second plasmid to either the third or fourth plasmid is greater than 1:1, when normalized to plasmid size. In some aspects, the molar ratio of the first plasmid to the fourth plasmid and the molar ratio of the second plasmid to the fourth plasmid are comparable when normalized to plasmid size. In certain aspects the molar ratio of the Rep plasmid and Cap plasmid are comparable when normalized to plasmid size.

In various aspects the method of lysing the transfected cells comprises at least one step selected from the group comprising a freeze-thaw step, a sonication step, a thermo-chemical step and an alternative chemical method step.

In an aspect, the titer of the harvested rAAV is at least 5×10¹⁰ vg/ml. In an aspect, the titer of the harvested rAAV is at least 1×10¹¹ vg/ml. In other aspects, the titer of the harvested rAAV is at least 3×10¹¹ vg/ml.

In various aspects of the systems, the step of transfecting HEK293-derived cells with said set of plasmids is co-transfection of the set of plasmids. In other aspects, the step of transfecting HEK293-derived cells with the plasmids of said set occurs sequentially.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 provides a chart summarizing the titer of a recombinant virus (rAAV2.7 m8-Fluc) in viral genomes per ml (vg/ml) produced from the indicated HEK293-derived cells after co-transfection with the indicated transfection reagent. Co-transfection of the AAV ITR-expression vector, pAAV Rep-Cap genes, and pHelper occurred in the presence of either AAV-Max (TRX) transfection reagent or PEIPro (PEIPRO) transfection reagent. The plasmids were transfected into either VPC2.0 cells (VPC2.0) or Oxford cells (OX). The data for the columns labeled OX-TRX-Molar, OX-PEIPro-Molar, VPC2.0-TRX-Molar and VPC2.0-PEIPro-Molar was obtained from experiments in which the cells were co-transfected with pAAV ITR-expression vector, pAAV Rep-Cap genes, and pHelper respectively in 1:2:0.5 molar ratio normalized to the plasmid size. The recombinant AAV titer obtained with the VPC2.0-PEIPro-Molar conditions in this series of experiments was greater than 5×10¹⁰ vg/ml. The recombinant AAV titer obtained with the VPC2.0-TRX-Molar conditions in this series of experiments was at least 1×10¹¹ vg/ml. The data for the column labeled OX-mass was obtained from experiments using equal molar concentrations of the Rep-Cap plasmid, the helper plasmid and the gene of interest (GOI) plasmids (1:1:1).

FIG. 2 provides a chart summarizing rAAV titers (y-axis, vg/ml) obtained from Oxford cells co-transfected with pAAV ITR-expression vector, pAAV Rep-Cap genes, and pHelper in the presence of PEIPro transfection reagent after lysis by the method indicated on the x axis. Results obtained from cells lysed by a freeze-thaw method (FT), a sonication method (SC), a thermo-chemical method (Thermo) or an alternative chemical method (ΔLT) are shown.

FIG. 3 provides a chart summarizing rAAV titers obtained from VPC2.0 cells co-transfected with pAAV ITR-expression vector, pAAV Rep-Cap genes, and pHelper at the indicated molar ratio normalized to plasmid size.

FIG. 4 provides a chart summarizing the rAAV titers obtained from the indicated cell type (Oxford (OX) or VPC2.0 (VPC2.0)) co-transfected with the indicated transfection reagent (PElpro or TRX). The same gene of interest or transgene was inserted in the same backbone, but the transgene was linked to either the 886 signal peptide (886, SEQ ID NO:1) or the 1176 signal peptide (1176, SEQ ID NO: 2). The 1176 construct with a different signal peptide resulted in higher titers.

FIG. 5 provides a schematic depiction of a three-plasmid system for rAAV production in two different types of HEK-derived cells (Oxford or VPC2.0).

FIG. 6 provides a chart summarizing the rAAV titers produced in VPC2.0 cells after co-transfection with either a 3-plasmid system (3plasmids) or a 4-plasmid system (4plasmids). In the conventional 3-plasmid system, cells were co-transfected with pAAV ITR-expression vector, pAAV Rep-Cap genes, and pHelper. In the 3-plasmid system, the Rep and Cap genes are on the same plasmid. In the 4-plasmid system, cells were co-transfected with pAAV ITR-expression vector, pAAV Rep genes, pAAV-CMV-Cap genes, and pHelper. The Rep genes and Cap genes were on separate plasmids. The titer increase obtained from the four plasmid system is substantial.

FIG. 7 provides a schematic depiction of a three plasmid system and a four plasmid system for rAAV production in HEK-derived cells.

### DETAILED DESCRIPTION

Methods and systems for producing high-titer rAAV are provided herein. The methods allow production of rAAV at substantially higher titer than typically achieved by standard production processes.

The following passages describe different aspects of the invention in greater detail. Each aspect, embodiment, or feature of the invention may be combined with any other aspect, embodiment, or feature the invention unless clearly indicated to the contrary.

### Definitions

Unless defined otherwise, all technical and scientific terms used herein have the meaning commonly understood by a person skilled in the art to which this invention belongs.

The terminology used herein is for the purpose of describing particular examples only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing” and is inclusive or open-ended. Any reference to “or” herein is intended to encompass “and/or” unless otherwise stated.

As used herein, the term “about” a number refers to that number plus or minus 10% of that number. The term “about” a range refers to that range minus 10% of its lowest value and plus 10% of its greatest value. Reference to “about” a value or parameter herein includes (and describes) embodiments that are directed to that value or parameter per se.

“High-titer” is intended to the concentration of particles or virions is at or above a pre-determined threshold prior to dilution; viral preparations with a lower titer resulting from dilution or use of a greater volume to harvest the particles or virions are encompassed by the term “high-titer” if the pre-determined threshold was obtainable at harvest. Titer type may be selected from, but is not limited to, total capsid titer, the genome titer, the physical titer, the infectious titer of the virus preparation, and the ratio of the full versus empty capsids. It is recognized that different titer types are assessed using different methods and the value of each titer type for a particular aliquot of viral particles may differ. It is understood that a “High-titer” preparation need only have a high concentration of particles for at least one titer-type. The terms “genome titer” and the “physical titer” are used interchangeably herein. Genome titer is understood to be the concentration of viral genomes per ml (vg/ml). Methods of evaluating genome titer are known in the art and include, but are not limited to, quantitative real-time PCR (qPCR) and digital droplet PCR (ddPCR). The total capsid titer represents the amount of all intact (fully assembled) viral capsids in a sample, including both the full and empty capsids. Methods of evaluating capsid titer are known in the art and include, but are not limited to, antibody-based methods, ELISAs, and dot blots. Methods of determining the ratio of full vs empty particles are known in the art and include but are not limited to cryo-electron microscopy, negative-staining, and combinations of methods to determine genome titer and capsid titer. See for example Gimpel et al (2021) Mol Therapy: Methods & Clin Dev 20:740-754. Pre-determined thresholds for “high-titer” rAAV include but are not limited to 5×10¹⁰ vg/ml, 5.5×10¹⁰ vg/ml, 6×10¹⁰ vg/ml, 6.5×10¹⁰ vg/ml, 7×10¹⁰ vg/ml, 7.5×10¹⁰ vg/ml, 8×10¹⁰ vg/ml, 8.5×10¹⁰ vg/ml, 9×10¹⁰ vg/ml, 9.5×10¹⁰ vg/ml, 1×10¹¹ vg/ml, 1.5×10¹¹ vg/ml, 2×10¹¹ vg/ml, 2.5×10¹¹ vg/ml, 3×10¹¹ vg/ml, 3.5×10¹¹ vg/ml, 4×10¹¹ vg/ml, 4.5×10¹¹ vg/ml, 5×10¹¹ vg/ml, 5.5×10¹¹ vg/ml, 6×10¹¹ vg/ml, 6.5×10¹¹ vg/ml, 7×10¹¹ vg/ml, 7.5×10¹¹ vg/ml, 8×10¹¹ vg/ml, 8.5×10¹¹ vg/ml, 9×10¹¹ vg/ml, 9.5×10¹¹ vg/ml, 1×10¹² vg/ml, and 5×10¹² vg/ml. High titer rAAV may have a concentration in the range of at least approximately 5×10¹⁰ vg/ml, 5.5×10¹⁰ vg/ml, 6×10¹⁰ vg/ml, 6.5×10¹⁰ vg/ml, 7×10¹⁰ vg/ml, 7.5×10¹⁰ vg/ml, 8×10¹⁰ vg/ml, 8.5×10¹⁰ vg/ml, 9×10¹⁰ vg/ml, 9.5×10¹⁰ vg/ml, 1×10¹¹ vg/ml, 1.5×10¹¹ vg/ml, 2×10¹¹ vg/ml, 2.5×10¹¹ vg/ml, 3×10¹¹ vg/ml, 3.5×10¹¹ vg/ml, 4×10¹¹ vg/ml, 4.5×10¹¹ vg/ml, 5×10¹¹ vg/ml, 5.5×10¹¹ vg/ml, 6×10¹¹ vg/ml, 6.5×10¹¹ vg/ml, 7×10¹¹ vg/ml, 7.5×10¹¹ vg/ml, 8×10¹¹ vg/ml, 8.5×10¹¹ vg/ml, 9×10¹¹ vg/ml, 9.5×10¹¹ vg/ml, 1×10¹² vg/ml, and 5×10¹² vg/ml.

In some embodiments, the methods and systems of the application may be used to yield an rAAV virion for use in a subject. In various aspects, the subject refers to a mammal. Mammals include, but are not limited to, domesticated animals (e.g. cows, sheep, cats, dogs, and horses), primates (e.g. humans and non-human primates such as monkeys), rabbits and rodents. In certain embodiments a “subject”, “patient”, or an “individual” refers to primates, such as humans and non-human primates, e.g., African green monkeys and rhesus monkeys. In some embodiments, the subject is a human.

The terms “treat,” “treating”, “treatment,” “ameliorate” or “ameliorating” and other grammatical equivalents as used herein, refer to alleviating, abating or ameliorating a disease or disorder, or symptoms of the disease or disorder, preventing additional symptoms of the disease or disorder, ameliorating or preventing the underlying metabolic causes of symptoms, inhibiting the disease or disorder, e.g., arresting the development of the disease or disorder, relieving the disease or disorder, causing regression of the disease or disorder, or stopping the symptoms of the disease or disorder, and are intended to include prophylaxis. The terms further include achieving a therapeutic benefit and/or a prophylactic benefit. The term “therapeutic benefit” refers to eradication or amelioration of the disease or disorder being treated. Also, a therapeutic benefit is achieved with the eradication or amelioration of one or more of the physiological symptoms associated with the disease or disorder such that an improvement is observed in the subject, notwithstanding that, in some embodiments, the subject is still afflicted with the disease or disorder. For prophylactic benefit, the pharmaceutical compositions are administered to a subject at risk of developing the disease or disorder, or to a subject reporting one or more of the physiological symptoms of the disease or disorder, even if a diagnosis of the disease or disorder has not been made.

The terms “administer,” “administering”, “administration,” and the like, as used herein, can refer to the methods that are used to enable delivery of therapeutics or pharmaceutical compositions to the desired site of biological action.

The terms “effective amount”, “therapeutically effective amount” or “pharmaceutically effective amount” as used herein, can refer to a sufficient amount of at least one pharmaceutical composition or compound being administered which will relieve to some extent one or more of the symptoms of the ocular disease or disorder being treated. An “effective amount”, “therapeutically effective amount” or “pharmaceutically effective amount” of a pharmaceutical composition may be administered to a subject in need thereof as a unit dose (as described in further detail elsewhere herein).

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

An “AAV vector” or “rAAV vector” as used herein refers to an adeno-associated virus (AAV) vector or a recombinant AAV (rAAV) vector comprising a polynucleotide sequence not of AAV origin (e.g., a polynucleotide heterologous to AAV such as a nucleic acid sequence that encodes a therapeutic transgene, e.g., aflibercept) for transduction into a target cell or to a target tissue. In general, the heterologous polynucleotide is flanked by at least one, and generally by two, AAV inverted terminal repeat sequences (ITRs). The term rAAV vector encompasses both rAAV vector particles and rAAV vector plasmids. A rAAV vector may be either single-stranded (ssAAV) or self-complementary (scAAV).

An “AAV virus” or “AAV viral particle” or “rAAV vector particle” or “rAAV particle” or “rAAV virion” or “rAAV virus” refers to a viral particle comprising at least one AAV capsid protein and a polynucleotide rAAV vector. In some cases, the at least one AAV capsid protein is from a wild type AAV or is a variant AAV capsid protein (e.g., an AAV capsid protein with an insertion, e.g., an insertion of the 7m8 amino sequence as set forth below). If the particle comprises a heterologous polynucleotide (e.g., a polynucleotide other than a wild-type AAV genome such as a transgene to be delivered to a target cell or target tissue), it is referred to as a “rAAV particle”, “rAAV vector particle” or a “rAAV vector”. Thus, production of rAAV particles necessarily includes production of a rAAV vector, as such a vector contained within a rAAV particle.

The term “packaging” as used herein can refer to a series of intracellular events that can result in the assembly and encapsidation of a rAAV particle.

AAV “rep” and “cap” genes refer to polynucleotide sequences encoding replication and encapsidation proteins of adeno-associated virus and variants thereof. The Rep genes encode the Rep proteins Rep78, Rep68, Rep52, and Rep40. Rep78 and Rep68 are transcribed from the p5 promoter, and Rep52 and Rep40 are transcribed from the p19 promoter. The Rep79 and Rep68 proteins are multifunctional DNA binding proteins that perform helicase and nickase functions during productive replication to allow for the resolution of AAV termini (see e.g., Im et al 1990 Cell 61:447-57. These proteins also regulate transcription from endogenous AAV promoters and promoters within helper viruses (see e.g., Periera et al (1997) J. Virol 71:1079-1088). The other Rep proteins modify the function of Rep78 and Rep68. In the four-plasmid system of the current application Rep may be operably linked to an AAV promoter including but not limited to p19 and p5. “Rep” encompasses variant Rep, chimeric Rep, and modified Rep.

The Cap genes encode the capsid proteins VP1, VP2 and VP3. The Cap genes are transcribed from the p40 promoter. In the four-plasmid system of the current application, Cap is operably linked to a constitutively active promoter. Cap encompasses variant Cap, chimeric Cap, pseudotyped Cap and modified Cap. One skilled in the art would be aware of which Rep and Cap forms are preferred for use together and would select appropriately.

The term “polypeptide” can encompass both naturally occurring and non-naturally occurring proteins (e.g., a fusion protein), peptides, fragments, mutants, derivatives and analogs thereof. A polypeptide may be monomeric, dimeric, trimeric, or polymeric. Further, a polypeptide may comprise a number of different domains, each of which has one or more distinct activities. For the avoidance of doubt, a “polypeptide” may be any length greater two amino acids.

As used herein, “polypeptide variant” or simply “variant” refers to a polypeptide whose sequence contains an amino acid modification. In some embodiments, the modification is an insertion, duplication, deletion, rearrangement or substitution of one or more amino acids compared to the amino acid sequence of a reference protein or polypeptide, such as a native or wild type protein. A variant may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino or carboxy termini. A variant can have the same or a different biological activity compared to the reference protein, or the unmodified protein.

In some embodiments, a variant can have, for example, at least about 80%, 85%, 90%, 91%, 92%, 93%, 94%, 95%, 96%, 97%, 98%, or 99% overall sequence homology to its counterpart reference protein. In some embodiments, a variant can have at least about 90% overall sequence homology to the wild-type protein. In some embodiments, a variant exhibits at least about 95%, at least about 98%, at least about 99%, at least about 99.5%, or at least about 99.9% overall sequence identity.

As used herein, “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell.

“Operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

The term “expression vector” or “expression construct” or “cassette” or simply “vector” can include any type of genetic construct, including AAV or rAAV vectors, containing a nucleic acid or polynucleotide coding for a gene product in which part or all of the nucleic acid encoding sequence is capable of being transcribed and is adapted for gene therapy. The transcript can be translated into a protein. In some embodiments, the transcript is partially translated or not translated. In certain aspects, expression includes both transcription of a gene and translation of mRNA into a gene product. In other aspects, expression only includes transcription of the nucleic acid encoding genes of interest. An expression vector can also comprise control elements operatively linked to the encoding region to facilitate expression of the protein in target cells. The combination of control elements and a gene or genes to which they are operably linked for expression can sometimes be referred to as an “expression cassette,” a large number of which are known and available in the art or can be readily constructed from components that are available in the art.

A “gene product” is a molecule resulting from expression of a particular gene. Gene products include, e.g., a polypeptide, an aptamer, an interfering RNA, a mRNA (messenger RNA), and the like. In particular embodiments, a “gene product” is a polypeptide, peptide, protein or interfering RNA including short interfering RNA (siRNA), miRNA (microRNA) or small hairpin RNA (shRNA). In particular embodiments, a gene product is a therapeutic gene product, e.g., a therapeutic protein. In some aspects, the gene product provides for knock-down of a gene function. In some aspects, the gene product provides for an increase of a gene function.

In some embodiments, the gene of interest may encode a reporter gene product such as, but not limited to, GFP and RFP. In some embodiments, the gene of interest may encode a therapeutic gene product such as a therapeutic protein. Therapeutic gene products are known in the art and include, but are not limited to, a polypeptide hormone, cytokine or growth factor (e.g. insulin or erythropoietin), an interferon, a blood clotting factor, a vaccine, an anti-angiogenic polypeptide, a vascular endothelial growth factor (VEGF) binding protein, an anti-VEGF agent, an anti-VEGF protein, an opsin protein, an anti-C3 antibody, an anti-C5 antibody, a hormone receptor (such as but not limited to mineralcorticosteroid, glucocorticoid, and thyroid hormone receptors), intramembrane proteins (such as but not limited to TM-1 and TM-7), intracellular receptors (such as but not limited to orphans, retinoids, vitamin D3 and vitamin A receptors), signaling molecules (such as but not limited to kinases, transcription factors and signal transducers and activators of transcription receptors of the cytokine superfamily (e.g. erythropoietin, growth hormone, interferons, interleukins and colony stimulating factors); G-protein coupled receptors such as but not limited to hormones, calcitonin, epinephrine, gastrin, paracrine or autocrine mediators such as somatostatin or prostaglandins; neurotransmitter receptors (norepinephrine, dopamine, serotonin or acetylcholine); ligands of tyrosine kinase receptors such as insulin growth factor and nerve growth factor; and an anti-dry AMD gene product. Anti-VEGF agents are known in the art and include, but are not limited to, bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept.

The term “anti-VEGF agent” includes any therapeutic agent, including proteins, polypeptides, peptides, fusion protein, multimeric proteins, gene products, antibody, human monoclonal antibody, antibody fragment, aptamer, small molecule, kinase inhibitor, receptor or receptor fragment, or nucleic acid molecule, that can reduce, interfere with, disrupt, block and/or inhibit the activity or function of an endogenous VEGF and/or an endogenous VEGF receptor (VEGFR), or the VEGF-VEGFR interaction or pathway in vivo. An anti-VEGF agent can be any one of the known therapeutic agents that can reduce new blood vessel growth or formation and/or edema, or swelling, when delivered into a cell, tissue, or a subject in vivo, e.g., ranibizumab, brolucizumab, or bevacizumab. In some embodiments, an anti-VEGF agent can be naturally occurring, non-naturally occurring, or synthetic. In some embodiments, an anti-VEGF agent can be derived from a naturally occurring molecule that was subsequently modified or mutated to confer an anti-VEGF activity. In some embodiments, an anti-VEGF agent is a fusion or chimeric protein. In such proteins, functional domains or polypeptides are artificially fused to a moiety or a polypeptide to make a fusion or chimeric protein that can sequester VEGF in vivo or function as a VEGFR decoy. In some embodiments, an anti-VEGF agent is a fusion or chimeric protein that blocks endogenous VEGFR from interacting with its ligands.

As used herein, “VEGF” can refer to any isoform of VEGF, unless required otherwise, including, but not limited to, VEGF-A, VEGF-B, VEGF-C, VEGF-D, VEGF-α, VEGF-F, or any combination, or any functional fragment or variant thereof. Unless required otherwise, “VEGF” can refer to any member of the VEGF family, including members: VEGF-A, placenta growth factor (PGF), VEGF-B, VEGF-C, and VEGF-D, or any combination, functional fragment, or variant thereof. As used herein, “VEGF receptor” or “VEGFR” or “VEGF-R” can be used to refer to any one of the receptors of VEGF, including, but not limited to, VEGFR-1 (or Flt-1), VEGFR-2 (or Flk-1/KDR), and VEGFR-3 (or Flt-4). VEGFR can be a membrane bound or soluble form, or a functional fragment or truncation of a receptor.

Therapeutic gene products for use in treating an ocular disease or disorder may include, but are not limited to, an anti-angiogenic polypeptide, a VEGF binding protein, an opsin protein, an anti-C3 antibody, an anti-C5 antidbody, an anti-dry AMD gene product, aflibercept, sFLT-1, CFI, ranibizumab and bevacizumab. An anti-dry AMD gene product may include, but is not limited to, inhibitors of C3, C5, HtrA1, Clqm, and natural inhibitors of the complement pathway, such as CFI, CFH and CD59.

As used herein, a “therapeutic gene” refers to a gene that, when expressed produces a therapeutic gene product that confers a beneficial effect on the cell or tissue when it is present or on a mammal in which the gene is expressed. Examples of beneficial effects include amelioration of a sign or symptom of a condition or disease, prevention or inhibition of a condition or disease, or conferral of a desired characteristic. Therapeutic genes include, but are not limited to, genes that corrects a genetic deficiency in a cell or mammal and genes that express a therapeutic gene product.

The term “heterologous” can refer to an entity that is genotypically distinct from that of the rest of the entity to which it is being compared. For example, a polynucleotide introduced by genetic engineering techniques into a plasmid or vector derived from a different species can be a heterologous polynucleotide. A promoter removed from its native coding sequence and operatively linked to a coding sequence with which it is not naturally found linked can be a heterologous promoter.

The methods, systems and kits described herein may employ, unless otherwise indicated, conventional techniques and descriptions of molecular biology (including recombinant techniques), cell biology, biochemistry, immunochemistry and virology techniques which are within the skill of those who practice the art. Such conventional techniques include methods for cloning and propagating recombinant virus, formulation of a pharmaceutical composition and biochemical purification and immunochemistry. Specific illustrations of suitable techniques can be had by reference to the examples herein. However, equivalent conventional procedures may also be used. Such conventional techniques and descriptions can be found in standard laboratory manuals such as Weiner et al., Eds, Genetic Variation: a Laboratory Manual (2007); Dieffenbach, Dveksler (Eds.), PCR Primer: a Laboratory Manual (2003); Sambrook and Russell, Condensed Protocols from Molecular Cloning: a Laboratory Manual (2006), all of which are hereby incorporated by reference in their entirety for all purposes.

Natural and engineered serotypes may be used in the methods and systems provided herein. Natural and engineered serotypes may include, but are not limited to AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), avian AAV, bovine AAV, canine AAV, equine AAV, primate AAV, non-primate AAV, rhesus macaque AAV, ovine AAV, AAV2.7m8, AAVrh10, AAVr100 and LSV1. Several AAV serotypes and over 100 AAV variants have been isolated from adenovirus stocks or from human or nonhuman primate tissues (reviewed in, e.g., Wu et al (2006) Molecular Ther.14 (3): 316-327. Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-5 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity and ability to establish long-term transgene expression (see, e.g., Carter, BJ (2005) Hum Gene Ther 16:541-550 and Wu et al. Genome sequences of various AAV serotypes and comparisons thereof are disclosed in, for example, GenBank Accession numbers U89790, J01901, AF043303 and AF085716; Chiorini et al. (1997) J. Virol 71:6823-33, Srivastava et al (1983) J. Virol. 45:555-564, Chiorini et al. (1999) J. Virol 73:1309-1319, Rutledge et al. (1998) J. Virol 72:309-319, and Wu et al. (2000) J. Virol 74:8635-8647.

In some embodiments, an AAV capsid protein is a wild-type AAV capsid protein, including but not limited to, an AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), avian AAV, bovine AAV, canine AAV, equine AAV, primate AAV, non-primate AAV, rhesus macaque AAV, or ovine AAV capsid protein. In some embodiments an AAV capsid protein is a VP1, VP2 or VP3 capsid protein. In some embodiments an AAV capsid protein is a hybrid of multiple AAV capsid proteins. A “variant AAV capsid protein” as used herein refers to an AAV capsid protein where the AAV capsid protein comprises at least one amino acid difference (e.g., amino acid substitution, amino acid insertion, amino acid deletion) relative to a corresponding parental AAV capsid protein, where the AAV capsid protein does not correspond to the amino acid sequence present in a naturally occurring AAV capsid protein. In various aspects, a variant AAV capsid protein may confer altered tropism to a recombinant AAV virion as compared to the tropism of a recombinant AAV virion comprising a parental AAV capsid protein. See US2020/0338146, herein incorporated by reference in its entirety.

In some embodiments, an AAV Rep protein is a wild-type AAV Rep protein, including but not limited to, an AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), avian AAV, bovine AAV, canine AAV, equine AAV, primate AAV, non-primate AAV, rhesus macaque AAV, or ovine AAV Rep protein. In some embodiments an AAV Rep protein is selected from the group comprising Rep78 protein, Rep68 protein, Rep52 protein and Rep40 protein. In some embodiments an AAV Rep protein is a hybrid of multiple AAV Rep proteins. A “variant AAV Rep protein” as used herein refers to an AAV Rep protein where the AAV Rep protein comprises at least one amino acid difference (e.g., amino acid substitution, amino acid insertion, amino acid deletion) relative to a corresponding parental AAV Rep protein, where the AAV Rep protein does not correspond to the amino acid sequence present in a naturally occurring AAV Rep protein.

A “modified sequence” as used herein refers to a sequence comprising one or more substitutions, insertions, and/or deletions as compared to the corresponding sequence of the parental sequence.

A “helper virus” for AAV refers to a virus that allows AAV (e.g., wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex virus (HSV) and Epstein-Barr viruses (EBV) as well as cytomegaloviruses (CMV) and pseudorabies virus (PRV).

AAV requires expression of helper genes for efficient replication. “Helper function(s)” refer to function(s) encoded in a helper virus genome which allow AAV replication and packaging (in conjunction with other requirements for replication and packaging). As described herein, “helper function” may be provided in a number of ways including but not limited to providing helper virus or providing for example a Helper plasmid comprising polynucleotide sequences encoding the requisite functions to a producer cell in trans. Requisite functions include but are not limited to the functions provided by the Adeno VA, E4 and E2A genes. For example, a plasmid or other expression vector comprising nucleotide sequences encoding one or more adenoviral helper proteins may be transfected or co-transfected into a producer cell. Cao et al 2000 J. Virol 74:11456-11463 described a method of producing high-titer, wild-type free recombinant AAV vectors using helper plasmids.

By “plasmid” is intended a small often circular extrachromosomal DNA molecule that can replicate independently in a cell. A plasmid may comprise one or more expression cassettes, open reading frames, polynucleotide cassettes, or expression vectors.

### Polynucleotides and Cells

Also provided herein are polynucleotides encoding one or more AAV capsid proteins. In particular embodiments the polynucleotide is an expression vector. In some embodiments the expression vector comprises the polynucleotide encoding a capsid protein, a modified capsid protein or a variant capsid protein operably linked to a promoter sequence that drives expression of the polynucleotide in a cell. In certain embodiments, the cell is a host cell. The host cell may be used to produce virions comprising the capsid protein. By “production cell” is intended a host cell used to produce rAAV virions. Exemplary host cells include mammalian cells including, but not limited to, HEK293 cells, HEK293-derived cells, VPC2.0 cells (a HEK293-derived cell) and Oxford cells (a HEK293-derived cell). In some embodiments, the polynucleotide comprising the polynucleotide encoding Cap further comprises a polynucleotide encoding Rep. In some embodiments, the polynucleotide comprising the polynucleotide encoding Rep is separate from the polynucleotide comprising the polynucleotide encoding Cap.

A “promoter” is a region of DNA that initiates transcription of a particular gene. Promoters from a wide variety of sources are well known in the art, and any promoter known in the art may be utilized in the methods and systems of the application. Promoters may be unidirectional (i.e., initiate transcription in one direction) or bi-directional (i.e., initiate transcription in either a 3′ or 5′ direction). Promoters include, but are not limited to, constitutively active promoters, inducible promoters and cell-type specific promoters. Constitutively active promoters include, but are not limited to, human beta-actin, chicken beta-actin, cytomegalovirus (CMV), SV40 and the CAG promoter. Cell-type specific promoters include but are not limited to the CD19 gene promoter, CaMKII1 and UAS. Inducible promoters include, but are not limited to the Tet system (U.S. Pat. Nos. 5,464,758 and 5,814,618), the ecdysone inducible system (No et al., (1996) Proc. Natl Acad Sci 93:3346-3351, the Cre-Lox system, the T-Rex™ system (Invitrogen, Carlsbad CA), the Cre-ERT tamoxifen inducible recombinase system (Indra et al (1999) Nuc Acid Res 27:4324-4327, U.S. Pat. No. 7,112,715, Kramer & Fussenegger 2005 Methods Mol Biol 308:123-144 and the LacSwitch™ system (Stratagene San Diego CA).

In certain embodiments the promoter operably linked to Cap is a constitutively active promoter. In an aspect, the promoter operably linked to Cap is a CMV promoter. It is recognized that a plasmid comprising the CMV promoter operably linked to Cap may further comprising a promoter operably linked to Rep. In certain embodiments, the promoter operably linked to Cap is a p40 promoter.

In some embodiments, a polynucleotide cassette comprising a sequence that encodes a gene product such as but not limited to a therapeutic gene product is provided. In various aspects the polynucleotide cassette is flanked by one or more AAV inverted terminal repeats (ITRs). In certain embodiments the polynucleotide cassette is flanked on the 5′ and 3′ ends by AAV ITRs. In some embodiments, the one or more ITRs are AAV1, AAV2, AAV3, AAV4, AAV5, AAV6, AAV7, AAV8, AAV9, AAV10, avian AAV, bovine AAV, canine AAV, equine AAV, primate AAV, non-primate AAV, rhesus macaque AAV, or ovine AAV ITR's or variants thereof. In some embodiments, the one or more ITRs are AAV1, AAV2, AAV3, AAV4, AAV5, AAV6, AAV7, AAV8, AAV9, AAV10, avian AAV, bovine AAV, canine AAV, equine AAV, primate AAV, non-primate AAV, rhesus macaque AAV, or ovine AAV ITR's. In some embodiments, the one or more ITRs are AAV1, AAV2, AAV3, AAV4, AAV5, AAV6, AAV7, AAV8, AAV9, AAV10, avian AAV, bovine AAV, canine AAV, equine AAV, primate AAV, non-primate AAV, rhesus macaque AAV, or ovine AAV ITR comprising one or more insertions, deletions and/or substitutions of nucleotides. An ITR expression vector comprises one or more of the following nucleic acid elements: a first ITR sequence; a promoter sequence; an intron sequence; a first UTR sequence; a gene of interest encoding a gene product; a second UTR sequence; a polyA sequence; and a second ITR sequence. In some embodiments, linker sequence(s) are inserted between two or more of the nucleic acid elements. In some embodiments, the heterologous nucleic acid encodes a therapeutic polypeptide, e.g., encodes aflibercept (or a functional fragment or functional variant thereof).

The terms “transgene”, “gene of interest” and “sequence that encodes a gene product” may be used interchangeably. The size of the transgene may affect the titer of the harvested rAAV as discussed below herein. Without being limited by mechanism, adjusting or altering the transgene size may increase the titer of harvested rAAV. The transgene size may be altered by deletion of one or more portions of the gene product or increased by addition of one or more filler regions. A filler region is a polynucleotide sequence of a size selected to improve rAAV titer including but not limited to a polynucleotide sequence encoding a signal peptide or a polynucleotide sequence encoding a cleavable sequence.

rAAV Production

Numerous methods are known in the art for production of recombinant AAV virions, including transfection, stable cell line production, and infectious hybrid virus production systems which include adenovirus-AAV hybrids, herpesvirus-AAV hybrids and baculovirus-AAV hybrids. rAAV production cultures for the production of rAAV virus particles all require 1) suitable production cells, 2) suitable helper functions, 3) AAV rep and cap genes; 4) a nucleic acid (such as a sequence encoding a transgene) flanked by at least one AAV ITR sequence and 5) suitable media and media components to support rAAV production. In some embodiments, the suitable production cell is a primate host cell. In some embodiments, the suitable host cell is a human-derived cell line such as a Hela, a549, HEK293 or Perc.6 cells. In a preferred embodiment the host cell is a HEK293-derived cell. In particularly preferred embodiments, the host cell is a VPC2.0 cell.

In the current methods and systems, the suitable helper functions are provided on a plasmid construct. An AAV helper construct comprises a polynucleotide comprising suitable helper function encoding sequences. Suitable helper function encoding sequences may include, but are not limited to the Adeno VA, E4 and E2A helper encoding sequences.

In some embodiments, the AAV rep gene may be from any AAV serotype or may be a modified AAV rep gene. In some aspects, the AAV rep gene is of the same serotype as the ITRs of the rAAV vector genome. In some aspects, the AAV rep gene is of a different serotype than the ITRs of the rAAV vector genome or the Cap serotype. In some aspects the AAV Rep is a chimeric Rep. In some aspects the AAV Cap is a chimeric Cap. In the three-plasmid system and methods using three plasmids, Rep and Cap are provided on the same plasmid. In the four-plasmid system and in methods using four plasmids, Rep and Cap are provided on different plasmids.

Suitable media known in the art may be used for the production of rAAV vectors. These media include, but are not limited to, Gibco Viral Production Medium supplemented with 4 mM GlutaMAX, media produced Hyclone Laboratories and JRH including Modified Eagle Medium (MEM), and Dulbecco's Modified Eagle Medium (DMEM).

Suitable rAAV production culture media may be supplemented with serum or serum-derived recombinant proteins at a level of 0.5%-20% (v/v or w/v). Alternatively, rAAV virions may be produced in serum-free conditions which may also be referred to as media with no animal-derived products. Commercial or custom media designed to support production of rAAV virions may also be supplemented with one or more cell culture components known in the art including, but not limited to, glucose, vitamins, amino acids, and growth factors to increase the titer of rAAV in production cultures.

Any transfection reagent are known in the art may be utilized in the methods and systems of the application. Transfection reagents may include, but are not limited to calcium phosphate, AAV-Max (also known as Trx) and PEI-Pro. The terms “Trx”, “TRX”, “AAV-MAX” and “AAV-Max” may be used interchangeably.

By “incubating” is intended keeping the transfected HEK293-derived cells in conditions including at a suitable temperature so that the cells develop and multiply. Methods of incubating HEK293-derived cells are known in the art. The transfected HEK293-derived cells may be incubated in media containing a reagent such as an antibiotic to which one or more of the plasmids confer resistance. The transfected HEK293-derived cells may be incubated in the media containing multiple selective reagents.

rAAV production cultures may be grown under a variety of conditions (over a wide temperature range, for varying lengths of time and the like) suitable for the particular host cell being utilized. rAAV production cultures include attachment dependent cultures and attachment independent cultures. Production cultures may be cultured in attachment dependent vessels including but not limited to roller bottles, hollow fiber filters, microcarriers, packed-bed bioreactors, fluidized-bed bioreactors and suspension-adapted vessels including but not limited to spinner flasks, stirred tank bioreactors and disposable systems including but not limited to the Wave bag system.

Also provided herein are compositions comprising rAAV's produced by a method or system of the current application. Pharmaceutical compositions comprising rAAV's produced by a method or system of the current application are provided.

One method for producing rAAV virions is the triple transfection method. Briefly, a plasmid containing the Rep gene and Cap gene, along with a helper adenoviral plasmid, and a plasmid comprising a transgene flanked by AAV ITRs may be co-transfected into a HEK293-derived cell line, incubated and virions collected and optionally purified. Transfection and co-transfection methods are known in the art and include, but are not limited to, calcium phosphate transfection.

“Co-transfecting” is intended to encompass concurrent transfection, simultaneous transfection, near concurrent transfection, near simultaneous transfection and rapid sequential transfection of multiple plasmids. Co-transfection of a three-plasmid system is challenging. Successful co-transfection of more than one plasmid may occur at a lower rate than successful transfection of a single plasmid. The challenges of successful co-transfection increase as the number of plasmids to be transfected increases. Without being limited by mechanism the challenges of co-transfection may relate to transfection efficiency of plasmids, selection of co-transfected cells and maintenance of multiple plasmids within co-transfected cells. In various aspects, transfecting the plasmids of the plasmid set occurs sequentially.

The current application provides a four plasmid system for producing rAAV. Briefly, first plasmid containing AAV Rep, a second plasmid containing AAV Cap, a third plasmid comprising an AAV helper construct, and a fourth plasmid comprising a transgene flanked by AAV ITRs may be transfected into HEK293-derived cells, the transfected cells are incubated, the transfected cells are lysed and rAAV are harvested and optionally purified. Transfection and co-transfection methods are known in the art and include, but are not limited to, calcium phosphate transfection.

It is recognized that the four plasmid system may be utilized in a modified producer cell line method, such as described in Martin et al. (2013) Human Gene Therapy Methods 24:253-269; US Pub No. US2004/0224411 and Liu, et al. (1999) Gene Ther. 6:293-299. Briefly the Rep gene and the Cap gene are provided on separate plasmids. It is understood that the producer cell line may contain either the Rep plasmid, the Cap plasmid or both the Rep plasmid and the separate Cap plasmid. In some embodiments a cell line stably transfected with a plasmid maintains the plasmid for multiple passages. The plasmid may replicate as the cell replicates or the plasmid may integrate into the cell genome.

In some embodiments, the plasmid(s) may contain a selectable marker (e.g., an antibiotic resistance marker) that allows for selection of cells maintaining the plasmid. Selectable markers include, but are not limited to, blasticidin, G418, hygromycin B, zeocin and puromycin. Methods for introducing nucleic acids into cells are known in the art and include without limitation viral transduction, cationic transfection, calcium phosphate transfection, microinjection, particle bombardment, electroporation and nanoparticle transfection. See, for example, Kim & Eberwine (2010) Anal Bioanal Chem 397:3173-3178.

In systems and methods comprising both the three-plasmid aspect and the four-plasmid aspect, it is recognized that the molar ratio of the plasmids when normalized to sized affects process yield. In various embodiments the molar ratios of the plasmids are approximately equivalent. In certain embodiments, the molar ratios of the plasmids may be approximately 1:1:1, 1:2:1, 1:3:1, 1:1:2, 2:1:1, 1:4:1, 1:4:2, 2:1:1, 2:1:2, 3:2:1, or 1:2:3 (helper plasmid: rep cap plasmid: ITR plasmid), when normalized to size. In certain embodiments, the molar ratio of the plasmids may be approximately 1:1:1:1 (helper plasmid: rep plasmid: cap plasmid: ITR plasmid). In various embodiments the molar ratio of the Cap Rep plasmid to either the helper plasmid or the plasmid comprising the ITR expression vector is in the range of 1:1, 1.5:1, 2:1, 2.5:1, 3:1, 3.5:1, 4:1, 4.5:1, 5:1 or more when normalized to plasmid size. In various embodiments the molar ratio of a Cap plasmid (second plasmid) to either the helper plasmid (third plasmid) or the plasmid comprising the ITR expression vector (fourth plasmid) is in the range of 1:1, 1.5:1, 2:1, 2.5:1, 3:1, 3.5:1, 4:1, 4.5:1, 5:1 or more when normalized to plasmid size. In various embodiments the molar ratio of a Rep plasmid (first plasmid) to either the helper plasmid (third plasmid) or the plasmid comprising the ITR expression vector (fourth plasmid) is in the range of approximately 1:1, 1.5:1, 2:1, 2.5:1, 3:1, 3.5:1, 4:1, 4.5:1, 5:1 or more when normalized to plasmid size. In various embodiments the molar ratio of a Rep plasmid (first plasmid to a Cap plasmid (second plasmid) is in the range of approximately 0.5:1, 1:1, 1.5:1, 2:1, 2.5:1, 3:1, 3.5:1, 4:1, 4.5:1, 5:1, 1:5, 1:4.5, 1:4, 1:3.5, 1:3, 1:2.5, 1:2, 1:1.5, 1:1, and 1:0.5 when normalized to plasmid size.

The terms “harvested” and “collected” may be used interchangeably. Methods of harvesting virions, viral particles and rAAV are known in the art and may include, but are not limited to filtration, flow filtration, depth filtration, membrane filtration, centrifugation, and combinations thereof. Any method of harvesting rAAV known in the art may be used in the methods and systems of the application.

Ocular diseases and disorders are known in the art. Ocular diseases and disorders include, but are not limited to, achromatopsia, glaucoma, retinitis pigmentosa, macular degeneration, retinoschisis, Leber's Congenital Amaurosis, diabetic retinopathy, color blindness, diabetic macular edema, choroidal neovascularization, proliferative diabetic retinopathy, retinal vein occlusion, central retinal vein occlusion, branched retinal vein occlusion, diabetic macular edema, diabetic retinal ischemia, ischemic retinopathy, diabetic retinal edema. Macular degeneration may include, but is not limited to, dry macular degeneration, wet macular degeneration, age-related macular degeneration and acute macular degeneration.

Age-related macular degeneration (AMD) is a degenerative ocular disease affecting the macula, a light sensitive, small area in the center of the retina that is responsible for reading and high acuity. Conditions affecting the macula reduce central vision while leaving peripheral vision intact. In severe cases, the disease can lead to central blindness. AMD is a notable cause of vision loss in the US population among persons 65 years and older, and the estimated prevalence of any AMD among persons over 40 years of age is approximately 6.5% (Klein et al., (2011) Arch Ophthalmol, 129 (1): 75-80).

There are two forms of age-related macular degeneration, dry (atrophic) and wet macular degeneration. Dry-AMD is more common than wet-AMD, but the dry can progress to wet-AMD. Dry-AMD is characterized by thinning of the tissues of the macula as cells disappear; dry-AMD may affect both eyes. Dry AMD is typically characterized by progressive apoptosis of the cells in the retinal pigment epithelium (RPE) layer, overlying photoreceptor cells, and frequently also the underlying cells in the choroidal capillary layer. Confluent areas of RPE cell death accompanied by overlying photoreceptor atrophy are referred to a geographic atrophy (GA). As dry-AMD progresses and GA increases, central vision slowly worsens and the ability to see fine detail is gradually lost. Dry AMD tends to progress more slowly than wet AMD.

In some embodiments, the ocular neovascular disease is recurrent and/or persistent wAMD. In some embodiments, the ocular neovascular disease is active subfoveal CNV secondary to AMD. In some embodiments, the active subfoveal CNV secondary to AMD occupies≥50% of the total lesion size. In some embodiments, the active subfoveal CNV secondary to AMD occupies≥50% of the total lesion size with evidence of leakage on fluorescein angiogram (FA), fluid on spectral domain optical coherence tomography (SD-OCT), and/or subretinal hemorrhage on color fundus photography. In some embodiments, the active subfoveal CNV secondary to AMD occupies≥50% of the total lesion size with evidence of leakage on fluorescein angiogram (FA), fluid on spectral domain optical coherence tomography (SD-OCT), and/or subretinal hemorrhage on color fundus photography, and the entire dimension of the lesion does not exceed 12 macular photocoagulation study disc areas. In some embodiments, the one eye and/or the contralateral eye of the individual exhibited best corrected visual acuity (BCVA) based on an ETDRS letters assessment of 78-25 (e.g., less than any of about 78, about 75, about 70, about 65, about 60, about 55, about 50, about 45, about 40, about 35, about 30, or about 25) prior to administration of the unit dose of rAAV particles of the present disclosure. In some embodiments, the one eye and/or the contralateral eye of the individual exhibited best corrected visual acuity (BCVA) based on an ETDRS letters assessment of more than any of about 5, about 10, about 15, about 20, about 25, about 30, about 35, about 40, about 45, about 50, about 55, about 60, about 65, about 70, about 75, about 80, about 85, about 90, about 95, or about 100 prior to administration of the unit dose of rAAV particles of the present disclosure.

In some embodiments, the individual had polypoidal choroidal vasculopathy (PCV) in the one eye and/or the contralateral eye prior to administration of the unit dose of rAAV particles.

Achromotopsia is a rare autosomal recessive disease that results in retinal degeneration affecting all three types of cone photoreceptor cells that results in reduced visual acuity, photophobia, hemeralopia, and severe loss of color discrimination. Mutation in the CNGB3 genes accounts for greater than 90% of patient and results in complete Achromatopsia, meaning that they have significant impairment in color discrimination and central visual acuity.

In some embodiments, a unit dose of rAAV particles is administered in combination with steroid treatment. In some embodiments, the steroid treatment is a corticosteroid treatment. In some embodiments, the steroid treatment is a systemic steroid treatment. In some embodiments, the steroid treatment is an oral steroid treatment. In some embodiments, the steroid treatment is a prednisone treatment. In some embodiments, the steroid treatment is an ophthalmic steroid treatment. In some embodiments, the ophthalmic steroid treatment is a topical steroid treatment (e.g., a drop), a periocular steroid treatment (e.g., subtenons, subconjunctival), an intravitreal steroid treatment, or a superchoroidal steroid treatment. In some embodiments, the ophthalmic steroid treatment is a glucocorticoid including, but not limited to, an anti-inflammatory glucocorticoid. In some embodiments, the topical steroid treatment is a glucocorticoid including but not limited to, an anti-inflammatory glucocorticoid. In some embodiments, the topical steroid treatment is a difluprednate treatment, a medrysone treatment, a loteprednol treatment, a prednisolone treatment, a fluocinolone treatment, a triamcinolone treatment, a rimexolone treatment, a dexamethasone treatment, a fluorometholone treatment, a fluocinolone treatment, a rimexolone treatment, or a prednisone treatment. Anti-inflammatory glucocorticoids may include, but are not limited to, difluprednate, dexamethasone, prednisolone, triamcinolone, fluorometholone, rimexolone, fluocinolone, loteprednol and bioequivalents thereof. In some embodiments, the topical steroid treatment is a difluprednate treatment. By “dexamethasone” is intended dexamethasone, dexamethasone biosimilars, dexamethasone bioequivalents, and pharmaceutical compositions comprising dexamethasone, a dexamethasone biosimilar or a dexamethasone bioequivalent. Pharmaceutical compositions comprising dexamethasone include, but are not limited to, Ozurdex™, Maxidex™, Decadron™, Dexamethasone Intensol™, Ocu-Dex™, Dexycu™, Dextenza™ and Zodex™. Ozurdex™ is a pharmaceutical composition comprising dexamethasone. By “difluprednate” is intended difluprednate, difluprednate biosimilars, difluprednate bioequivalents, and pharmaceutical compositions comprising difluprednate, a difluprednate biosimilar or a difluprednate bioequivalent. Pharmaceutical compositions comprising difluprednate include, but are not limited to, Durezol™ and difluprednate emulsions. By “triamcinolone” is intended triamcinolone, triamcinolone biosimilars, triamcinolone bioequivalents, and pharmaceutical compositions comprising triamcinolone, a triamcinolone biosimilar or a triamcinolone bioequivalent. Pharmaceutical compositions comprising triamcinolone include, but are not limited to, Triesence™, Xipere™, and Trivaris™. In some embodiments, the steroid treatment is administered before, during, and/or after administration of the unit dose of rAAV particles. In some embodiments, the steroid treatment is administered before administration of the unit dose of rAAV particles. In some embodiments, the steroid treatment is administered during administration of the unit dose of rAAV particles. In some embodiments, the steroid treatment is administered after administration of the unit dose of rAAV particles. In some embodiments, the steroid treatment is administered before and during administration of the unit dose of rAAV particles. In some embodiments, the steroid treatment is administered before and after administration of the unit dose of rAAV particles. In some embodiments, the steroid treatment is administered during, and after administration of the unit dose of rAAV particles. In some embodiments, the steroid treatment is administered before, during, and after administration of the unit dose of rAAV particles.

In some embodiments, the steroid treatment is an ophthalmic steroid treatment (e.g., difluprednate). In some embodiments, the ophthalmic steroid treatment (e.g., difluprednate) is a daily steroid treatment for up to about 4 weeks, about 6 weeks, or about 8 weeks from administering the unit dose of rAAV particles. In some embodiments, the ophthalmic steroid treatment comprises about four administrations of ophthalmic steroid on about week 1, about three administrations of ophthalmic steroid on about week 2, about two administrations of ophthalmic steroid on about week 3, and about one administration of ophthalmic steroid on about week 4; timing starting with and following administration of the unit dose of rAAV particles. In some embodiments, the ophthalmic steroid is about 0.005% to about 0.5% difluprednate. In some embodiments, the ophthalmic steroid is any of about 0.005%, about 0.006%, about 0.007%, about 0.008%, about 0.009%, about 0.01%, about 0.02%, about 0.03%, about 0.4%, about 0.05%, about 0.06%, about 0.07%, about 0.08%, about 0.09%, or about 0.1% difluprednate. In some embodiments, the ophthalmic steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some embodiments, the steroid treatment is an ophthalmic steroid treatment (e.g., difluprednate). In some embodiments, the ophthalmic steroid treatment (e.g., difluprednate) is a daily topical steroid treatment for up to about 4 weeks, about 6 weeks, or about 8 weeks from administering the unit dose of rAAV particles. In some embodiments, the topical steroid treatment comprises about four administrations of topical steroid on about week 1, about three administrations of topical steroid on about week 2, about two administrations of topical steroid on about week 3, and about one administration of topical steroid on about week 4; timing starting with and following administration of the unit dose of rAAV particles. In some embodiments, the topical steroid treatment comprises about four administrations of topical steroid (i.e., QID) per day for about 3 weeks after administration of the unit dose of rAAV particles, followed by about 3 administrations of topical steroid per day (i.e., TID) for about 1 week, followed by about 2 administrations of topical steroid per day (i.e., BID) for about 1 week, and followed by about 1 administration of topical steroid per day (i.e., QD) for about 1 week. In some embodiments, the topical steroid comprises difluprednate 0.05% at a dose of about 1 μg to about 3 μg. In some embodiments, the topical steroid comprises difluprednate 0.05% at a dose of about 2.5 μg. In some embodiments, the topical steroid is about 0.005% to about 0.5% difluprednate. In some embodiments, the topical steroid is any of about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% difluprednate. In some embodiments, the topical steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

It will be understood that the reference to the below examples is for illustration purposes only and do not limit the scope of the claims.

### EXAMPLES

### Example 1. AAV Production in HEK-Derived Cells

Cells are grown to a density of approximately 4.5 to 6.0×10⁶ viable cells/mL. Viability of ≥95% is confirmed prior to proceeding with transfection. Cells are diluted in medium supplemented with GlutaMax™ supplement (ThermoFisher). Immediately after dilution, AAV-Max or PEI-Pro is added to the dilute cell solution. The flask is swirled gently to mix the cells. The cells are incubated in a 37° C. incubator with a humidified atmosphere of 8% CO₂ on an orbital until the DNA/Transfection complexation process is completed. The transfer plasmid, Rep and Cap plasmid (or plasmids), and the helper plasmid DNA are prepared. The plasmid DNA molar ratio is optimized based on plasmid size, and approximately 1.5 g/mL of total plasmid DNA of culture volume to be transfected is used.

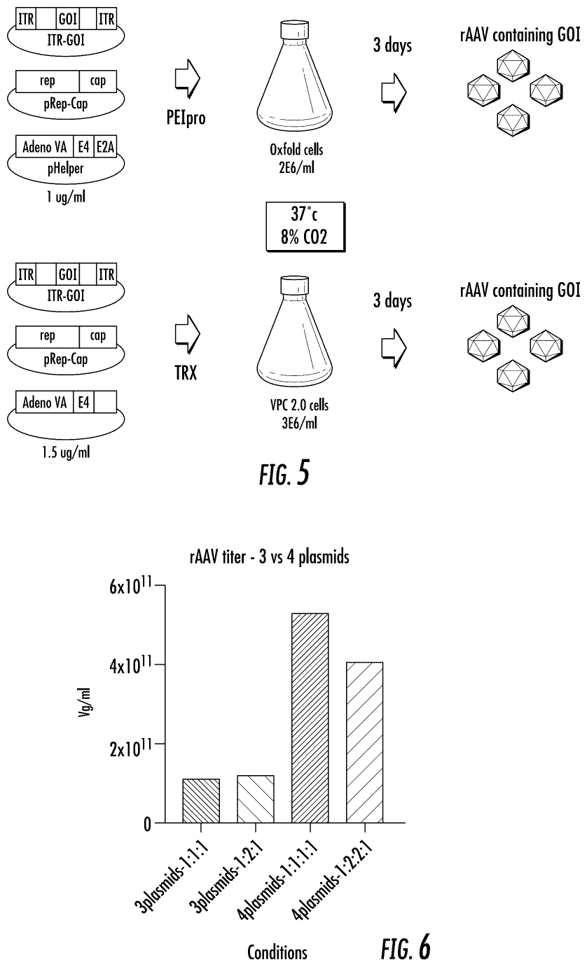

Transfection complexes are prepared as described below herein. The transfection reagent is gently mixed. The total plasmid DNA is diluted with complexation buffer. The diluted DNA is incubated at room temperature (RT) for 10 minutes. Transfection booster and transfection reagent are mixed gently and incubated together for 10 minutes at RT. The pre-mixed booster and reagent are mixed gently with the diluted plasmid DNA and incubated for 20-25 minutes at room temperature. The plasmid, booster and reagent combination is gently added to the shaker of cells. The cells are incubated in a 37° C. incubator with a humidified atmosphere of 8% CO₂ on an orbital shaker for approximately 72 hours. A cartoon schematic of the general process in two cell types is provided in FIG. 5.

### Example 2. AAV Production in VPC2.0 Cells

On the day of transfection (Day 0), viable cell density and percent viability of Gibco VPC2.0 cells (ThermoFisher Cat. No. A49784) was determined. Cells with a density of approximately 4.5 to 6.0×10⁶ viable cells/mL and viability≥95% proceeded to transfection. The cells were diluted to a final density of 3×10⁶ viable cells/mL with fresh Viral Production Medium supplemented with 4 mM GlutaMAX™ Supplement. Immediately after dilution, AAV-MAX was added. The flask was swirled gently to mix the cells and placed in a 37° C. incubator with a humidified atmosphere of 8% CO₂ on an orbital until DNA/Transfection complexation process was completed.

For three plasmid systems, transfer plasmid DNA, Rep/Cap plasmid DNA and Helper plasmid DNA were mixed using a molar ratio optimization based on the plasmid size. Approximately 1.5 g/ml of total plasmid DNA of culture volume to be transfected was prepared. Transfection complexes were prepared as described. The AAV-MAX Transfection Reagent bottle was mixed by gentle inversion. Total plasmid DNA was diluted with Viral-Plex™ Complexation Buffer to a final volume of 10% of the culture volume to be transfected. The plasmid DNA and Complexation Buffer was mixed gently and incubated at room temperature for 10 min.

Undiluted AAV-MAX Transfection Booster at 3 μL per mL of culture to be transfected followed by AAV-MAX Transfection Reagent at 6 μL per mL of culture to be transfected were added to a new tube. The Booster and Reagent were mixed gently and incubated at RT for 10 minutes. The mixed AAV-MAX Transfection Booster and AAV-MAX Transfection Reagent were added to the diluted plasmid DNA and mixed gently. The plasmid DNA/AAV-MAX Booster/AAV-MAX Reagent complexes were incubated at room temperature for 20 to 25 minutes and then gently transferred to the cell solution in the shaker flask. The cells were incubated in a 37° C. incubator with a humidified atmosphere of 8% CO2 on an orbital shaker for approximately 72 hours.

### Example 3 Harvest

AAV particles were harvested 70 to 72 hours post-transfection. AAV-MAX Lysis buffer was added directly to the culture flask at a 1:10 dilution. The flask was swirled to evenly distribute the lysis buffer. 1M MgCl2 was added to a final 2-4 mM concentration in culture. 90U/mL Benzonase was added to the flask. The flask was incubated at 37° C. for at least 2 hours on an orbital shaker. Cells were spun at 4200 RPM, 4° C. for 30 min and the supernatant was transferred to appropriate size container, and stored at −80° C.

### Example 4. Harvest by Freeze/Thaw or Sonication

Cells were counted to determine VCD, TCD, and % viability. The cells were collected in an appropriately sized vessel and centrifuged at 3500 RPM, 4° C. for 10 min. The supernatant was discarded. The cell pellet was resuspended in lysis buffer (0.15M NaCl, 50 mM Tris-Cl, 0.05% Tween-20; pH 8) at (˜1 mL/2.8E+7 cells). The cells were subjected to three rounds of freeze thaw cycles or sonication (approximately 100 ml batch). MgCL₂ was added to 2 mM. The lysate was treated with benzonase (50 ul/ml) for 1 hour. The cells were spun at 4200 RPM, 4° C. for 30 min. The supernatant was transferred to an appropriately sized container and stored at 4° C. or −80° C.

When sonication was used, the resuspended cells were chilled on ice prior and post sonication. A Branson 250 Digital Sonifier with a flat tip was used at amplitude 50% with 1 second pulses on/off for 40 seconds.

### Example 5. Evaluation of Transfection Reagents

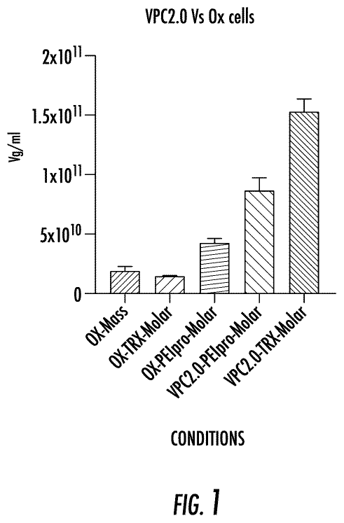

pAAV ITR-expression vector, pAAV Rep-Cap genes, and pHelper respectively in 1:2:0.5 molar ratio normalized to the plasmid size were co-transfected into either Oxford or VPC2.0 cells with either the PElpro® or TRX (AAV-MAX transfection reagent) transfection reagents. rAAV2.7m8-Fluc was harvested from the cells and the titer was determined by ddPCR as described elsewhere herein. Results from one such series of experiments are presented in FIG. 1.

### Example 6. Titer Determination

Titers were determined using ddPCR. The digested harvested rAAV was diluted 1/5000 folds by dilution buffer with 0.05% Pluronic F-68 (Gibco, Invitrogen, Grand Island, NY). The reaction mixtures were assembled using a ddPCR™ Supermix for Residual DNA detection kit (BIO-RAD, Hercules, CA) with 0.9 μM primers and 0.1 μM probe, in a final volume of 25 μl as stated in the manufacturer's protocol. ddPCR was done as described in ddPCR amplification guide (Bio-Rad 6407). Then, test samples were emulsified with droplet generator oil using a QX-100 droplet generator (BIO-RAD) according to the manufacturer's instructions. PCR amplification of the droplets was performed using a conventional thermal cycler with the following parameters: 95° C. for 10 min, followed by 39 cycles of 94° C. for 30 s and 60° C. for 30 s, followed by a final 98° C. heat treatment for 10 min. The PCR plate was subsequently scanned using a QX200 droplet reader (BIO-RAD) and the data were analyzed with QuantaSoft software (BIO-RAD). For 2D ddPCR, two sets of primers and probes labeled with 5 (6)-carboxyflourescein (FAM) and hexachloro-fluorescein (HEX), respectively, were added to each reaction mixture, and ddPCR was performed in the same tube.

### Example 7. Lysis Condition Evaluation

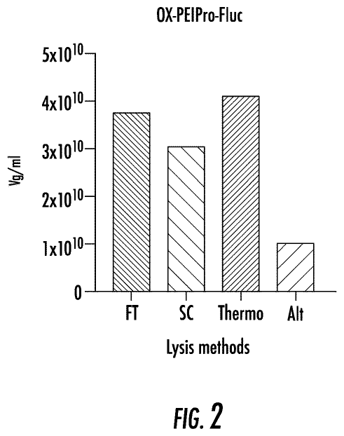

Various lysis conditions were evaluated. rAAV2.7m8-CMV-Fluc capsid was produced in Oxford cells with PElpro® transfection reagent. Oxford cells containing rAAVs were lysed with different lysis conditions: Freeze thaw (FT), Sonication (SC), Thermo chemical method (Thermo), and Alternative chemical method (Alt). The alternative chemical method buffer is 10 mM EDTA, 20 mM TRIS-Base, pH 9.2, Benzonase 10u/ml, MgSO4.7H2o 40 mM and 0.4M NaCl. Results from one such series of experiments are summarized in FIG. 2.

### Example 8. Plasmid Molar Ratio Optimization

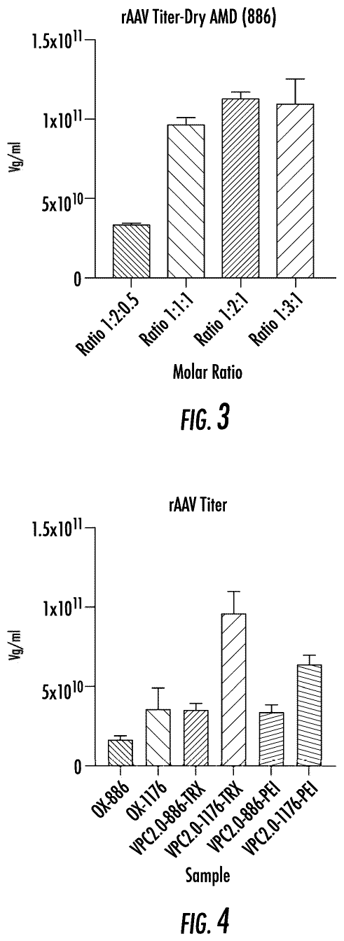

rAAV2.7m8-Dry AMD was produced in VPC2.0 cells by co-transfection of pAAV ITR-expression vector, pAAV Rep-Cap genes, and pHelper, respectively, in different molar ratios normalized to the plasmid size. Results from one such series of experiments are summarized in FIG. 3.

### Example 9. Impact of Transgene

rAAV with the same transgene inserted into the same backbone with two different signal peptides (886 and 1176) were produced and the titer of the harvested rAAV was determined. p886 has the IgK signal sequence (MDMRVPAQLLGLLLLWLRGARC, SEQ ID NO:1), whereas p1176 has a CD33 signal sequence (MPLLLLLPLLWAGALA, SEQ ID NO:2). rAAV2.7m8-Dry AMD (886 & 1176) were produced in Oxford and VPC2.0 cells with two different transfection reagents (PElpro® & TRX). Results from one such series of experiments are summarized in FIG. 4.

### Example 10. Comparison of Four Plasmid System and Three Plasmid System

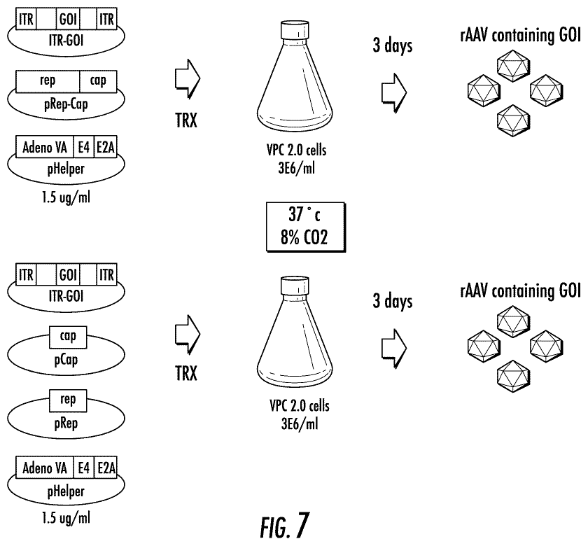

rAAV2.7m8-CMV-GFP was produced in VPC2.0 cells with two different transfection systems. First condition was a conventional three plasmid system by co-transfection of pAAV ITR-expression vector, pAAV Rep-Cap genes, and pHelper. The second condition was a four plasmid system. The pAAV Rep-Cap genes were split into two separate plasmids (a pAAV-Rep gene plasmid and a pAAV-CMV-Cap gene plasmid) in which the CMV promoter was used for the Cap genes. pAAV ITR-expression vector, pAAV Rep genes, pAAV-CMV-Cap genes, and pHelper were co-transfected into VPC2.0 cells as described elsewhere herein. The transfected cells were incubated. The rAAV2.7m8-CMV-GFP particles were harvested and the titers were determined by ddPCR. Results from one such series of experiments are summarized in FIG. 6.

Surprisingly the four plasmid system method increased the rAAV titer substantially. In at least one series of experiments, the rAAV2.7m8-CMV-GFP titer was increased more than five fold when a four plasmid system was used.

That which is claimed:

1. A method of producing high-titer rAAV comprising (a) providing HEK293-derived cells, (b) co-transfecting HEK293-derived cells with plasmids comprising an ITR expression vector, an AAV helper construct, Rep and Cap, (c) incubating the transfected cells and (d) harvesting the rAAV.

2. The method of claim 1, wherein Rep and Cap are on separate plasmids.

3. The method of claim 1, wherein Rep and Cap are on the same plasmid.

4. The method of claim 1, wherein the transfection reagent used in co-transfecting the cells is selected from the group comprising AAV-MAX and PEI-Pro.

5. The method of claim 1, wherein the HEK293-derived cells are selected from the group comprising VPC2.0 cells and Oxford cells.

6. The method of claim 1, wherein the HEK293-derived cells are selected from the group comprising VPC2.0 cells and Oxford cells and wherein the transfection reagent used in co-transfecting the cells is selected from the group comprising AAV-MAX and PEI-Pro.

7. The method of claim 6, wherein the HEK293-derived cells are VPC2.0 cells and the transfection reagent is AAV-Max.

8. The method of claim 1, wherein the titer of the harvested rAAV is at least 5×10¹⁰ vg/ml.

9. The method of claim 1, wherein the transfection reagent used in co-transfecting the cells is selected from the group comprising AAV-MAX and PEI-Pro, and wherein Rep and Cap are on separate plasmids.

10. The method of claim 9, wherein the transfection reagent is AAV-Max.

11. The method of claim 1, wherein the HEK293-derived cells are selected from the group comprising VPC2.0 cells and Oxford cells and wherein Rep and Cap are on separate plasmids.

12. The method of claim 11, wherein the HEK293-derived cells are VPC2.0 cells.

13. The method of claim 1, wherein the HEK293-derived cells are selected from the group comprising VPC2.0 cells and Oxford cells, wherein the transfection reagent used in co-transfecting the cells is selected from the group comprising AAV-MAX and PEI-Pro and wherein Rep and Cap are on separate plasmids.

14. The method of claim 13, wherein the HEK293-derived cells are VPC2.0 cells, the transfection reagent is AAV-Max and wherein Rep and Cap are on separate plasmids.

15. The method of claim 8, wherein the titer of the harvested rAAV is at least 1×10¹¹ vg/ml.

16. A four plasmid system for producing high titer rAAV comprising the steps of:
(a) providing a set of plasmids comprising a first plasmid comprising a nucleotide sequence encoding an AAV Rep polypeptide operably linked to a first promoter, a second plasmid comprising a nucleotide sequence encoding an AAV Cap polypeptide operably linked to a second promoter, a third plasmid comprising an ITR expression vector comprising a gene of interest, and a fourth plasmid comprising an AAV helper construct;
(b) transfecting HEK293-derived cells with said set of plasmids;
(c) incubating the transfected cells;
(d) lysing the transfected cells; and
(e) harvesting the rAAV.

17. The system of claim 16, wherein the second promoter is a constitutively active promoter.

18. The system of claim 17, wherein the constitutively active promoter is a CMV promoter.

19. The system of claim 16, wherein the second promoter is a p40 promoter.

20. The system of claim 16, wherein the first promoter is an AAV promoter.

21. The system of claim 16, wherein the AAV promoter is a p19 promoter.

22. The system of claim 16, wherein said HEK293-derived cells are selected from the group comprising HEK293 cells, VPC2.0 cells and Oxford cells.

23. The system of claim 16, wherein the molar ratio of the first plasmid to the third plasmid and the molar ratio of the second plasmid to the third plasmid are comparable when normalized to plasmid size.

24. The system of claim 23, wherein the four plasmids of said set of plasmids are co-transfected in molar ratios of approximately 1:1:1:1 when normalized to plasmid size.

25. The system of claim 16, wherein the molar ratio of the first plasmid to either the third or fourth plasmid is greater than 1:1, when normalized to plasmid size.

26. The system of claim 16, wherein the molar ratio of the second plasmid to either the third or fourth plasmid is greater than 1, when normalized to plasmid size.

27. The system of claim 16, wherein the molar ratio of the first plasmid to the fourth plasmid and the molar ratio of the second plasmid to the fourth plasmid are comparable when normalized to plasmid size.

28. The system of claim 16, wherein the method of lysing the transfected cells comprises at least one step selected from the group comprising a freeze-thaw step, a sonication step, a thermo-chemical step and an alternative chemical method step.

29. The system of claim 16, wherein the titer of the harvested rAAV is at least 5×10¹⁰ vg/ml.

30. The system of claim 29, wherein the titer of the harvested rAAV is at least 1×10¹¹ vg/ml.

31. The system of claim 30, wherein the titer of the harvested rAAV is at least 3×10¹¹ vg/ml.

32. The system of claim 16, wherein the step of transfecting HEK293-derived cells with said set of plasmids is co-transfection of the set of plasmids.

33. The system of claim 16, wherein the step of transfecting HEK293-derived cells with the plasmids of said set occurs sequentially.


SEQUENCE LISTING (2 sequences)

SEQ ID NO: 1 - protein, 22 aa, synthetic construct
MDMRVPAQLLGLLLLWLRGARC

SEQ ID NO: 2 - protein, 16 aa, synthetic construct
MPLLLLLPLLWAGALA